(19) United States
(12) Patent Application Publication
(10) Pub. No.: US 2011/0262997 A1
(43) Pub. Date: Oct. 27, 2011

(54) PRODUCTION OF CELLULASE

(75) Inventors: Mads Peter Torry Smith, Raleigh, NC (US); Guillermo Coward-Kelly, Wake Forest, NC (US)

(73) Assignee: Novozymes North America, Inc., Franklinton, NC (US)

(21) Appl. No.: 13/178,196

(22) Filed: Jul. 7, 2011

Related U.S. Application Data
(63) Continuation of application No. 11/917,627, filed on Dec. 14, 2007, now abandoned.
(60) Provisional application No. 60/695,722, filed on Jun. 30, 2005.

(51) Int. Cl. C12N 9/42

(52) U.S. Cl. 435/209

(57) ABSTRACT

The invention relates to a process of producing cellulase in a host cell, comprising cultivating said host cell capable of producing cellulase under conditions conducive for production of cellulase, wherein pre-treated ligno-cellulosic material is added to induce cellulase production. The invention also relates to use of pre-treated ligno-cellulosic material as inducer or carbon source in cellulase production processes.

### CROSS-REFERENCE TO RELATED APPLICATIONS

This application is a continuation of U.S. application Ser. No. 11/917,627 filed on Dec. 14, 2007, which is a 35 U.S.C. 371 national application of PCT/US2006/026110 filed Jun. 30, 2006, which claims priority or the benefit under 35 U.S.C. 119 of U.S. provisional application No. 60/695,722 filed Jun. 30, 2005, the contents of which are fully incorporated herein by reference.

### TECHNICAL FIELD

The present invention relates to a process of producing cellulase in a host cell in an economical way.

### BACKGROUND ART

Microbial host cells are use for producing cellulase. The largest fraction of feedstock costs are glucose (carbon source) and pure cellulose (inducer and carbon source). Addition of pure cellulose as inducer to stimulate cellulase production is well known in the art. Pure cellulose is available from commercial suppliers, but is expensive. Therefore, there is a need for providing an easily available and cheap inducer that can replace expensive pure cellulose used today.

### SUMMARY OF THE INVENTION

It is the object of the present invention to provide a process of producing cellulase in a host cell using an easily available and cheap inducer as replacement for expensive pure cellulose or a similar inducer.

According to the first aspect the invention relates to a process of producing cellulase in a host cell comprising cultivating said host cell capable of producing cellulase under conditions conducive for production of cellulase, wherein pre-treated ligno-cellulosic material is added to induce cellulase production. The host cell may be a recombinant or wild-type host cell as will be described further below.

The invention also relates to the use of pre-treated ligno-cellulosic material as inducer and/or carbon source for producing cellulase in a host cell.

### BRIEF DESCRIPTION OF THE DRAWING

FIG. 1 shows SDS-PAGE of APE-[57˜59].

FIG. 2 shows PCS hydrolysis vs. enzyme (relative protein in broth/g cellulose) loading. APE-58 is control fermentation with cellulose in batch.

FIG. 3 shows PCS hydrolysis vs. enzyme (relative volume broth/g cellulose) loading. APE-58 is control fermentation with cellulose in batch.

### DETAILED DISCLOSURE OF THE INVENTION

It is the object of the present invention to provide processes of producing cellulases in host cells using an easily available and cheap inducer material that can replace expensive cellulase inducer, e.g., pure cellulose, used today.

A standard feed for cellulase production is glucose feed with suspended pure cellulose.

The inventors have found that pure cellulose used today for cellulase production in a host cell, may be replaced with pre-treated ligno-cellulosic material, such as, especially pre-treated corn stover (PCS). Before use, the pre-treated ligno-cellulosic material is preferably detoxified, e.g., by washing, e.g., by repeated soaking in water, ion exchange, stripping and the like. The detoxification is done at least partly to remove compounds that inhibit the performance of the host cell. An advantage of the invention is that the production cost is reduced due to use of an inducer which is easily available and thus cheaper than pure cellulose.

### Process of Producing Enzymes

It is well known in the art to produce cellulase in a host cell of fungal origin, such as filamentous fungi, or bacteria origin. The process of the invention may be a well known process, except that the inducer, such as pure cellulose, is replaced by pre-treated ligno-cellulosic material.

A host cell capable of producing cellulase is grown under precise cultural conditions at a particular growth rate. When the host cell culture is introduced into the fermentation medium the inoculated culture pass through a number of stages. Initially growth does not occur. This period is referred to as the lag phase and may be considered a period of adaptation. During the next phase referred to as the “exponential phase” the growth rate of the host cell culture gradually increases. After a period of maximum growth the rate ceases and the culture enters stationary phase. After a further period of time the culture enters the death phase and the number of viable cells declines. Where in the growth phase the cellulase is expressed depends on the cellulase and host cell. The cellulase may in one embodiment be expressed in the exponential phase. In another embodiment the cellulase is produced in the transient phase between the exponential phase and the stationary phase. The cellulase may also in an embodiment be expressed in the stationary phase and/or just before sporulation. The cellulase may according to the invention also be produced in more that one of the above mentioned phases.

In other words, according to the invention the host cell is cultivated in a suitable medium and under conditions allowing cellulase to be expressed, preferably secreted and optionally recovered. The cultivation takes place in a fermentation medium comprising at least a carbon source and pre-treated ligno-cellulosic material as inducer. According to a preferred embodiment the inducer is washed pre-treated ligno-cellulosic plant material. Cellulase production procedures are well known in the art. In context of the present invention the cellulase is preferably an extra-cellular cellulase secreted into the fermentation medium by the host cell. Alternatively, the cellulase is intracellular. After fermentation the cellulase may optionally be recovered using methods well known in the art. For example, extra-cellular cellulase recovery from the fermentation medium may be done using conventional procedures including, but not limited to, centrifugation, filtration, extraction, spray-drying, evaporation, or precipitation. Procedures for recovery of intracellular cellulase are also well known in the art.

At least in context of the present invention the interchangeable terms “cultivation” and “fermentation” means any process of producing cellulase using a mass culture consisting of one or more host cells. The present invention is useful for especially industrial scale production, e.g., having a culture medium of at least 50 litres, preferably at least 100 litres, more preferably at least 500 litres, even more preferably at least 1,000 litres, in particular at least 5,000 litres.

A process of the invention may be performed as a batch, a fed-batch, a repeated fed-batch or a continuous process.

A process of the invention may be carried out aerobically or anaerobically. Some enzymes are produced by submerged cultivation and some by surface cultivation. Submerged cultivation is preferred according to the invention.

Thus, according to the first aspect, the invention relates to processes of producing cellulase in a host cell comprising cultivating said host cell capable of producing cellulase under conditions conducive for production of cellulase, wherein pre-treated ligno-cellulosic material is added to induce cellulase production.

### Substrate

The substrate used in a process of the invention may be any substrate known in the art. Suitable substrates are available from commercial suppliers or may be prepared according to published compositions (e.g., in catalogues of the American Type Culture Collection).

Carbon source substrates commonly used for cellulase production includes glucose or similar sugars. Nitrogen source substrates, growth stimulators and the like may be added to improve cultivation and cellulase production. Nitrogen sources include ammonia (NH₄Cl) and peptides. Protease may be used, e.g., to digest proteins to produce free amino nitrogen (FAN). Such free amino acids may function as nutrient for the host cell, thereby enhancing the growth and cellulase production. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. Examples of minerals include minerals and mineral salts that can supply nutrients comprising P, K, Mg, S, Ca, Fe, Zn, Mn, and Cu.

According to the present invention pure cellulose, usually used as inducer (and carbon source) in cellulase production processes, is replaced with pre-treated ligno-cellulosic material, preferably detoxified, such as washed, pre-treated ligno-cellulosic material.

According to the invention the pre-treated ligno-cellulosic material may be added to the culture medium together with a carbon source, but may also be added separate from the carbon source. According to the invention the pre-treated ligno-cellulosic material may be added to the culture medium either prior to inoculation, simultaneously with inoculation or after inoculation of the host cell culture in an amount corresponding to the amount of pure cellulose normally used. This means that the pre-treated ligno-cellulosic material is preferably added in an amount that equals that of pure cellulose normally used. A person skilled in the art can easily determine when to add and which amount of pre-treated ligno-cellulosic material to add during a cellulase producing process of the invention. During the time span of cultivation pre-treated ligno-cellulosic material is preferably added in an amounts corresponding to that of pure cellulose normally used. In a preferred embodiment the ratio between the amount of pre-treated ligno-cellulosic material (corresponding to the amount of pure cellulose) and carbon source, such as glucose, lies in the range from about 1:10 to 2:1, preferably from about 1:5 to 1:1.

As mentioned above pre-treated ligno-cellulosic material is used the same way pure cellulose is normally used in well known cellulase production processes.

For instance, when producing cellulase using a strain of Trichoderma, such as Trichoderma reesei, as host cell the carbon source substrate level is kept low, e.g., below 1 g carbon source substrate/L, such as below 1 g glucose/L. A process of the invention may last for the same period of time as a corresponding traditional process, such as between 3 and 10 days. Trichoderma fermentations, including Trichoderma reesei fermentations, in general last for between 5-9 days.

### Ligno-Cellulosic Material

According to the invention “ligno-cellulosic material” includes any material that comprises ligno-cellulose. Ligno-cellulose is generally found, for example, in the stems, leaves, hulls, husks, and cobs of plants or leaves, branches, and wood of trees. The ligno-cellulosic material can also be, but is not limited to, herbaceous material, agricultural residues, forestry residues, municipal solid wastes, waste paper, and pulp and paper mill residues. It is understood herein that ligno-cellulosic material may be in the form of plant cell wall material containing lignin, cellulose, and hemi-cellulose in a mixed matrix.

In an embodiment the ligno-cellulosic material is corn fiber, rice straw, pine wood, wood chips, poplar, wheat straw, switch grass, bagasse, paper and pulp processing waste. In a preferred embodiment the ligno-cellulosic material is corn stover. In another preferred embodiment the ligno-cellulosic material is woody or herbaceous plants.

### Pre-Treatment

According to the invention ligno-cellulosic material is pre-treated. The term “pre-treated” may be replaced with the term “treated”. However, preferred techniques contemplated are those well known for “pre-treatment” of ligno-cellulosic material as will be describe further below.

As mentioned above treatment or pre-treatment may be carried out using conventional methods known in the art, which promotes the separation and/or release of cellulose from ligno-cellulosic material.

Pre-treatment techniques are well known in the art and include physical, chemical, and biological pre-treatment, or any combination thereof. In preferred embodiments the pre-treatment of ligno-cellulosic material is carried out as a batch or continuous process.

Physical pre-treatment techniques include various types of milling/comminution (reduction of particle size), irradiation, steaming/steam explosion, and hydrothermolysis.

Comminution includes dry, wet and vibratory ball milling. Preferably, physical pre-treatment involves use of high pressure and/or high temperature (steam explosion). In context of the invention high pressure include pressure in the range from 300 to 600 psi, preferably 400 to 500 psi, such as around 450 psi. In context of the invention high temperature include temperatures in the range from about 100 to 300° C., preferably from about 140 to 235° C. In a specific embodiment impregnation is carried out at a pressure of about 450 psi and at a temperature of about 235° C. In a preferred embodiment the physical pre-treatment is done using a steam gun hydrolyzer system which uses high pressure and high temperature, such as, using the Sunds Hydrolyzer (available from Sunds Defibrator AB (Sweden).

Chemical pre-treatment techniques include acid, dilute acid, base, organic solvent, lime, ammonia, sulfur dioxide, carbon dioxide, pH-controlled hydrothermolysis, wet oxidation, and solvent treatment.

Preferably, the chemical treatment process is an acid treatment process, more preferably, a continuous dilute or mild acid treatment, such as treatment with sulfuric acid, or another organic acid, such as acetic acid, citric acid, tartaric acid, succinic acid, or any mixture thereof. Other acids may also be used. Mild acid treatment means at least in the context of the invention that the treatment pH lies in the range from 1 to 5, preferably 1 to 3. In a specific embodiment the acid concentration is in the range from 0.1 to 2.0 wt % acid, preferably sulfuric acid. The acid is mixed or contacted with the ligno-cellulosic material and the mixture is held at a temperature in the range of around 160-220° C. for a period ranging from minutes to seconds. Specifically the pre-treatment conditions may be the following: 165-183° C., 3-12 minutes, 0.5-1.4% (w/w) acid concentration, 15-25, preferably around 20% (w/w) total solids concentration. Other contemplated methods are described in U.S. Pat. Nos. 4,880,473, 5,366,558, 5,188,673, 5,705,369 and 6,228,177 which are hereby all incorporated by reference.

Wet oxidation techniques involve the use of oxidizing agents, such as sulfite based oxidizing agents and the like. Examples of solvent treatments include treatment with DMSO (Dimethyl Sulfoxide) and the like. Chemical treatment processes are generally carried out for about 5 to about 10 minutes, but may be carried out for shorter or longer periods of time.

Biological pre-treatment techniques include applying lignin-solubilizing micro-organisms (see, for example, Hsu, T.-A., 1996, Pre-treatment of biomass, in Handbook on Bioethanol: Production and Utilization, Wyman, C. E., ed., Taylor & Francis, Washington, D.C., 179-212; Ghosh, P., and Singh, A., 1993, Physicochemical and biological treatments for enzymatic/microbial conversion of ligno-cellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolysates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95).

In an embodiment both chemical and physical pre-treatment is carried out including, for example, both mild acid treatment and high temperature and pressure treatment. The chemical and physical treatment may be carried out sequentially or simultaneously.

In a preferred embodiment the pre-treatment is carried out as a dilute acid steam explosion step. In another preferred embodiment the pre-treatment is carried out as an ammonia fiber explosion step (or AFEX pre-treatment step).

In an embodiment of the invention, e.g., dilute-acid hydrolyzed, ligno-cellulosic material, such as corn stover, is steam stripped in order to detoxify the material.

In a preferred embodiment the pre-treated ligno-cellulosic material consists essentially of cellulose.

### Cellulase

A cellulase means according to the invention a cellulolytic enzyme capable of degrading biomass. A cellulase produced according to the invention may be of any origin including of bacterial or fungal origin. Chemically modified or protein engineered variants are included. Suitable cellulases include cellulases from the genera Bacillus, Pseudomonas, Humicola, Fusarium, Thielavia, Acremonium, Chrysosporium and Trichoderma, e.g., fungal cellulases produced by Humicola insolens, Myceliophthora thermophila, Thielavia terrestris, Fusarium oxysporum, Chrysosporium lucknowense, and Trichoderma reesei.

In an embodiment the cellulase produced is a cellulase complex homologous to the host cell. In an embodiment the cellulase produced is a cellulase complex homologous to a host cell of the genus Trichoderma, preferably a strain of Trichoderma reesei.

In another preferred embodiment the cellulase is a cellulase preparation comprising the Trichoderma reesei cellulase complex and in addition thereto one or more foreign enzymes co-produced heterogously.

In another embodiment the cellulase produced is a cellulase complex homologous to a strain of the genus Humicola, preferably a strain of Humicola insolens, especially Humicola insolens, DSM 1800.

In another preferred embodiment the cellulase produced is a cellulase preparation comprising the Humicola insolens cellulase complex and in addition thereto one or more foreign enzymes co-produced heterogously.

In an embodiment the cellulase produced is the cellulase complex homologous to a strain of the genus Chrysosporium, preferably a strain of Chrysosporium lucknowense.

In another preferred embodiment the cellulase produced is a cellulase preparation comprising the Chrysosporium lucknowense cellulase complex and in addition thereto one or more foreign enzymes co-produced heterogously.

It is to be understood that the cellulase produced may also be a mono-component cellulase, e.g., an endoglucanase, exo-cellobiohydrolase, glucohydrolase, or beta-glucosidase produced recombinantly in a suitable host cell. Suitable host cells are described further below.

The cellulase produced may also be a cellulase preparation where one or more homologous cellulase components are deleted or inactivated from the host cell natively producing the cellulase.

### Host Cell Capable of Producing Cellulase

The host cell may be of any origin. As mentioned above the cellulase may be homologous or heterologous to the host cell capable of producing the cellulase.

The term “recombinant host cell”, as used herein, means a host cell which harbours gene(s) encoding cellulase and is capable of expressing said gene(s) to produce cellulase, wherein the cellulase coding gene(s) have been transformed, transfected, transducted, or the like, into the host cell. The transformation, transfection, transduction or the like technique used may be well known in the art. In a preferred embodiment the gene is integrated into the genome of the recombinant host cell in one or more copies.

When the cellulase is heterologous the recombinant host cell capable of producing the cellulase is preferably of fungal or bacterial origin. The choice of recombinant host cell will to a large extent depend upon the gene(s) coding for the cellulase and the origin of the cellulase.

The term “wild-type host cell”, as used herein, refers to a host cell that natively harbours gene(s) coding for cellulase and is capable of expressing said gene(s). When the cellulase is a homologous preparation or cellulase complex the wild-type host cell or mutant thereof capable of producing the cellulase is preferably of fungal or bacterial origin.

A “mutant thereof” may be a wild-type host cell in which one or more genes have been deleted or inactivated, e.g., in order to enrich the cellulase preparation in a certain component. A mutant host cell may also be a wild-type host cell transformed with one or more additional genes coding for additional enzymes or proteins in order to introduce one or more additional enzyme activities or other activities into the cellulase complex or preparation natively produced by the wild-type host cell. The additional enzyme(s) may have the same activity (e.g., cellulase activity) but merely be another enzyme molecule, e.g., with different properties. The mutant wild-type host cell may also have additional homologous enzyme coding genes transformed, transfected, transducted, or the like, preferably integrated into the genome, in order to increase expression of that gene to produce more enzyme.

In a preferred embodiment the recombinant or wild-type host cell is of filamentous fungus origin. Examples of host cells include the ones selected from the group comprising Acremonium, Aspergillus, Aureobasidium, Bjerkandera, Ceriporiopsis, Chrysosporium, Coprinus, Coriolus, Cryptococcus, Filobasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a more preferred embodiment the filamentous fungal host cell is selected from the group comprising a strain of Aspergillus awamori, Aspergillus fumigatus, Aspergillus foetidus, Aspergillus japonicus, Aspergillus nidulans, Aspergillus niger or Aspergillus oryzae. In another preferred embodiment the filamentous fungal host cell is a strain of Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another preferred embodiment, the filamentous fungal host cell is selected from the group comprising a strain of Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, or Ceriporiopsis subvermispora, Chrysosporium lucknowense Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride.

In another preferred embodiment the recombinant or wild-type host cell is of bacterial origin. Examples of host cells include the ones selected from the group comprising gram positive bacteria such as a strain of Bacillus, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

### Use

In the second aspect the invention relates to the use of pre-treated ligno-cellulosic material as inducer for producing cellulase in a host cell.

In the third aspect the invention relates to the use of pre-treated ligno-cellulosic material as carbon source in cellulase production processes.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure, including definitions will be controlling.

Various references are cited herein, the disclosures of which are incorporated by reference in their entireties.

### Materials & Methods

### Materials

Trichoderma reesei SMA135-04 is disclosed in example 8 of the US patent publication no. 2005/0233423.

Trace Metals Preparation

| Components | g/L |
|---|---|
| FeCl₃•6H₂0 | 216 |
| ZnSO₄•7H₂O | 58 |
| MnSO₄•H₂O | 27 |
| CuSO₄•5H₂O | 10 |
| H₃BO₃ | 2.4 |
| Citric Acid | 336 |

Seed Flask Preparation

| Components | g/L |
|---|---|
| Glucose | 20 |
| Corn Steep Solids | 10 |
| (NH₄)₂ SO₄ | 1.45 |
| KH₂PO₄ | 2.08 |
| CaCl₂•2H₂O | 0.36 |
| MgSO₄•7H₂O | 0.42 |
| Trace Metals (mL) | 0.2 |
| L61 Pluronic | (1-2) drops per SF |
| pH | 5 |
| Autoclave Time (min) | 30 |

Seed Flask Inoculation

| | |
|---|---|
| Inoculant (cm² of PDA Plate) | 1 |
| Seed Flask Volume (mL) | 100 |
| Incubation Time (hrs) | 45 |
| Shaker Temp | 28 |
| Shaker RPM | 200 |
| Transfer Criteria | pH < 4.0 |
| Vessel Inoculation Volume (mL) | 50 |

Post Sterile Additions

| | | Sterile | | | |
|---|---|---|---|---|---|
| Components | Frequency | Method | APE-57 | APE-58 | APE-59 |
| 1:5 L61 | Daily, | Autoclave | 10-100 mL/day | | |
| Pluronic | as needed | | | | |

### Example 1

### Utility of Pre-Treated Ligno-Cellulosic Material (Biomass) for Production of Cellulase Enzymes

In this example Trichoderma reesei was grown on washed biomass solids resulting from pre-treatment by heat and dilute acid. Pre-treated Corn Stover (PCS) was provided by the National Renewable Energy Laboratory (NREL, Golden Colo.) with glucan content of 53.2% (NREL data). 1 kg PCS was suspended in a ˜20 liter of double deionized water in a bucket and, after the PCS settled, the water was decanted. This was repeated until the wash water is above pH 4.0, at which time the reducing sugars was lower than 0.06 g/L. Percent dry weight content of the washed PCS was determined by drying the sample at a 105° C. oven for more than 24 hours (until constant weight) and comparing to the wet weight.

Fermentations were performed in Applikon 2 L glass jacketed vessels, which have a working volume of 1.8 L. Temperature was measured by electronic thermocouples and controlled using a circulating water bath. Dissolved oxygen and pH were both measured using sensor probes purchased from Broadley James Corporation. An ADI 1030 controller allowed proportional feedback control to adjust pH using acid and base feed pumps based on a pH set-point and deadband. ADI 1012 stirrer controllers were used to drive an Applikon P310 motor to agitate the broth at speeds ranging from 1100 to 1300 rpm. Rushton radial-flow impellers were utilized without baffles. The broth was aerated using a sterile air flow at a rate of about 1 vvm; the air entered via a sparger located at the bottom of the tank, beneath the impeller.

The fermentation was run using Trichoderma reesei strain SMA135-04. Glycerol freezer stocks have been prepared and were used as inoculum for the seed flasks. Seed flasks were grown as shown in the table below. Some inocula were reduced in volume as shown.

Trichoderma fermentation lasted for approximately 165 hours, at which time the tank was harvested. The Trichoderma fermentation method utilizes a glucose feed. Pluronic® L61 surfactant (BASF) is used to reduce foaming as necessary. Examples with PCS in batch (APE-57, APE-59) are compared to fermentation with cellulose in batch (APE-58).

Fermentation Medium

| | APE-57 | APE-58 | APE-59 |
|---|---|---|---|
| Components | g | g | g |
| Corn Steep Solids | 18.0 | 18.0 | 18.0 |
| Cellulose | | 75.0 | |
| PCS | 783.2 | | 783.2 |
| Glucose | 7.2 | 7.2 | 7.2 |
| CaCl₂•2H₂O | 4.8 | 4.8 | 4.8 |
| (NH₄)₂ SO₄ | 6.8 | 6.8 | 6.8 |
| KH₂PO₄ | 5.0 | 5.0 | 5.0 |
| MgSO₄•7H₂O | 2.9 | 2.9 | 2.9 |
| Trace Metals (mL) | 1.4 | 1.4 | 1.4 |
| L 61 Pluronic (mL) | 3.2 | 3.2 | 3.2 |
| Media Volume Added to Tank (L) | 1.8 | 1.8 | 1.8 |

| | |
|---|---|
| pH | 4.3 |
| Autoclave Time (min) | 60 |

Feed Composition

FEED COMPOSITION

| | APE-57 | APE-58 | APE-59 |
|---|---|---|---|
| Components | g | g | g |
| Glucose | 900.0 | 900.0 | 900.0 |
| H₂O | 592.5 | 592.5 | 592.5 |
| Cellulose | 0 | 0 | 0 |
| L61 Pluronic | 7.5 | 7.5 | 7.5 |

OPERATING CONDITIONS

| | |
|---|---|
| Starting Volume (L) | 1.8 |
| Temperature (° C.) | 28 |
| pH | 4.75 ± 0.1 |
| Starting Agitation (rpm) | 1100 |
| Air Flow (VVM) | 1 |
| Minimum DO (%) | 25 |

| | | Standard | APE-57 | APE-58 | APE-59 |
|---|---|---|---|---|---|
| Feed (g wet feed/hr) | 0 hrs | 0 | 0 | 0 | 0 |
| | 18 hrs | 3.6 | 1X | 1X | 1X |
| | 33 hrs | 7.2 | 1X | 1X | 1X |

| | | | |
|---|---|---|---|
| pH Control | Acid | 5N H₃PO₄ | 1X = standard feed rate in |
| | | | g glucose/hr |
| | Base | 28% NH₄OH | |

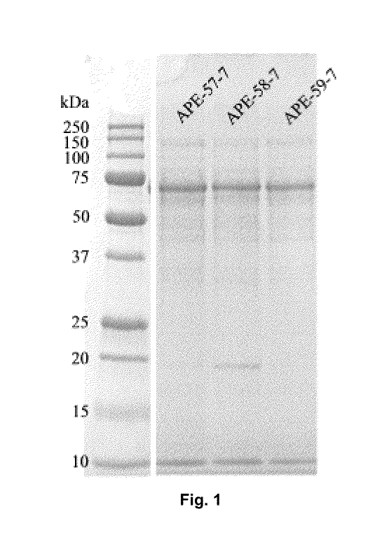

Aliquots of final fermentation broths were diluted 5 times in distilled deionised (DDI) water. Then 1 volume of diluted sample was mixed with 2 volume of SDS sample buffer (BioRad) mixed with 5% beta-mercaptoethanol, boiled for 5 minutes. 15 microL of each sample was loaded onto 8-16% Tris-HCl gel (BioRad), electrophoresed and stained with BioSafe Coomassie Blue (FIG. 1). The major bands of proteins are at ˜70 kDa. All broths showed a clear band at ˜10 kDa; cellulose in batch also showed a band at <18 kDa.

The activities of enzyme broths were measured by their ability to hydrolyze dilute-acid pre-treated corn stover (PCS) and produce sugars detectable by a chemical assay of their reducing ends. PCS was provided by the National Renewable Energy Laboratory (NREL, Golden Colo.) with glucan content of 53.2% (NREL data). 1 kg PCS was suspended in a ˜20 liter of double deionized water in a bucket and, after the PCS settled, the water was decanted. This was repeated until the wash water is above pH 4.0, at which time the reducing sugars was lower than 0.06 g/L. The settled slurry was sieved through 100 Mesh screens to ensure ability to pipette. Percent dry weight content of the washed PCS was determined by drying the sample at a 105° C. oven for more than 24 hours (until constant weight) and comparing to the wet weight.

PCS hydrolysis was performed in 96-deep-well plates (Axygen Scientific) sealed by a plate sealer (ALPS-300, ABgene). PCS concentration was 10 g/L, with 50 mM acetate pH 5.0. PCS hydrolysis was done at 50° C., with total reaction volume of 1.0 ml, without additional stirring. Each reaction was done in triplicates. Released reducing sugars were analyzed by p-hydroxy benzoic acid hydrazide (PHBAH) reagent as described below.

In detail, a 0.8 ml of PCS (12.5 g/L) was pipetted into each well of the 96-deep-well plates, to this 0.10 ml of sodium acetate buffer (0.5 M, pH 5.0) was added, then 0.10 ml diluted enzyme solution was added to start the reaction and to give the final reaction volume of 1.0 ml and PCS concentration of 10 g/L. The reaction mixture was mixed by inverting the deep-well plate at the beginning of hydrolysis and before taking each sample timepoint. After mixing, the deep-well plate was centrifuged (Sorvall RT7 with RTH-250 rotor) at 3000 rpm for 2 minutes before 20 microL of hydrolysate (supernatant) was removed and added to 180 microL of 0.4% NaOH in a 96-well microplate. This stopped solution was further diluted into the proper range of reducing sugars if necessary. The reducing sugars released were assayed by para-hydroxy benzoic acid hydrazide reagent (PHBAH, Sigma, 4-hydroxy benzyhydrazide): 50 microL PHBAH reagent (1.5%) was mixed with 100 microl sample in a V-bottom 96-well Thermowell plate (Costar 6511), incubated on a plate heating block at 95° C. for 10 min, then 50 microL DDI water was added to each well, mixed and 100 microL was transferred to another flat-bottom 96-well plate (Costar 9017) and absorbance read at 410 nm. Reducing sugar was calculated using a glucose calibration curve under the same conditions. Percent conversion of cellulose to reducing sugars was calculated as:

% conversion=reducing sugars (mg/ml)/(cellulose added (mg/ml)×1.11)

The factor 1.11 corrects for the weight gain in hydrolyzing cellulose to glucose.

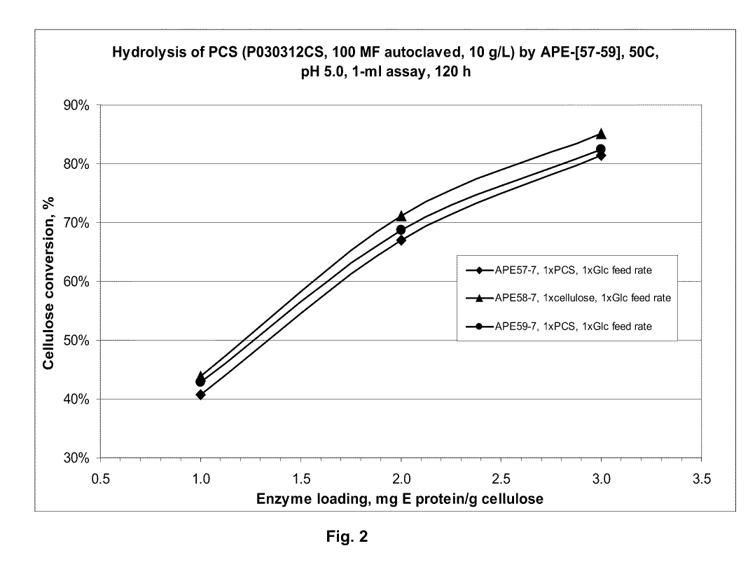

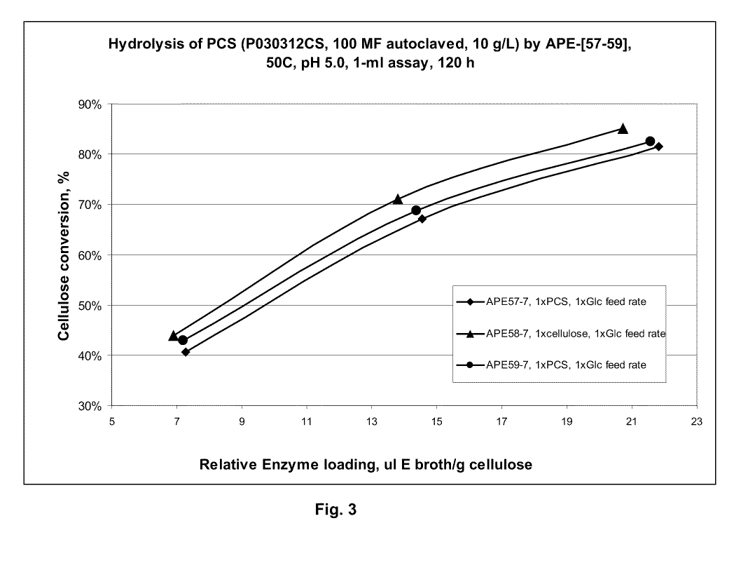

APE-57 and APE-59, using PCS, produced protein levels similar to control fermentations with cellulose (FIG. 3), as well as broth with similar cellulase activity to control fermentations with cellulose (FIG. 2).

1-19. (canceled)

20. A process of producing a cellulase, comprising cultivating a host cell capable of producing the cellulase under conditions conducive for the production of the cellulase, wherein pre-treated ligno-cellulosic material is added in an amount effective to induce cellulase production and glucose is added as the substrate for the host cell.

21. The process of claim 20, wherein the ligno-cellulosic material is plant material.

22. The process of claim 21, wherein the plant material is selected from the group comprising: corn stover, corn fiber, rice straw, pine wood, wood chips, poplar, wheat straw, switch grass, paper, and pulp processing waste.

23. The process of claim 20, wherein the pre-treatment of the ligno-cellulosic material is carried out by subjecting the ligno-cellulosic material to physical treatment, chemical treatment, biological treatment, or any combination thereof.

24. The process of claim 20, wherein the pre-treated ligno-cellullosic material has been subjected to milling, dilute acid steam explosion, steam explosion, wet oxidation, or ammonia fiber explosion (or AFEX pre-treatment).

25. The process of claim 20, wherein the host cell is a recombinant host cell or wild-type host cell or a mutant thereof.

26. The process of claim 20, wherein the host cell is of bacterial or fungal origin.

27. The process of claim 20, wherein the cellulase is a cellulase complex or preparation derived from of the genus Chrysosporium, Humicola, or Trichoderma.

28. The process of claim 20, wherein the cellulase is a cellulase complex or preparation derived from Chrysosporium lucknowense, Humicola insolens, or Trichoderma reesei.

29. The process of claim 20, wherein the cellulase is a mono-component cellulase derived from Aspergillus, Chrysosporium, Humicola, or Trichoderma.

30. The process of claim 20, wherein the cellulase is a mono-component cellulase derived from Aspergillus niger, Aspergillus oryzae, Chrysosporium lucknowense, Humicola insolens, or Trichoderma reesei.

31. The process of claim 20, which takes place in a fermentation tank of at least 50 liters.

32. The process of claim 20, which is carried out as a fed-batch process.

33. The process of claim 20, wherein the glucose is added before, together with, or after the pre-treated ligno-cellulosic material.

34. The process of claim 20, further comprising adding a nitrogen source.

35. The process of claim 20, wherein the pre-treated material consists essentially of cellulose.

36. The process of claim 20, further comprising recovering the cellulase.

37. The process of claim 20, wherein the pre-treated ligno-cellulosic material is detoxified before being used for cellulase production.
